(19) United States
(12) Patent Application Publication
(10) Pub. No.: US 2022/0266049 A1
(43) Pub. Date: Aug. 25, 2022

(54) Handheld LED therapy device

(71) Applicant: Kinreen Technology (Shenzhen) Co., Ltd ., Shenzhen (CN)

(72) Inventors: Fly Zhang, Shenzhen (CN); Robin Yi, Shenzhen (CN); Polo Cheng, Shenzhen (CN); Jane Jiang, Shenzhen (CN); Wendy Hao, Shenzhen (CN); Marry Gong, Shenzhen (CN); Coco Wen, Shenzhen (CN)

(21) Appl. No.: 17/183,250

(22) Filed: Feb. 23, 2021

(51) Int. Cl. A61N 5/06

(52) U.S. Cl. CPC A61N 5/06; A61N 2005/0652; A61N 2005/0663; A61N 2005/0629; A61N 2005/0644

(57) ABSTRACT

A handheld LED therapy device, includes a main controlling module and an LED therapy module. The LED therapy module includes a multilight cup comprising a LED therapy light of at least one light color, the main controlling module is connected to the LED therapy light, and the main controlling module can operate a controlling operation to the LED therapy light. The present disclosure emits light of at least one color, to beautify the skin of human body, promote the metabolism and circulation of subcutaneous tissue, increase collagen, accelerate the repair of damaged tissues, relieve pain, accelerate cell renewal and repair, functioning in therapy, meeting people's requirements of beauty therapy effects and vision.

### TECHNICAL FIELD

The present disclosure relates to the technical field of beauty therapy, and in particular to a handheld LED therapy device.

### BACKGROUND

A LED therapy device uses the principle of photo-dynamics to activate deep cells, facilitating the metabolism of skin. After the light is absorbed by the skin, the light energy is converted into intracellular energy, which relaxes and strengthens the capillaries and produces a photo-chemical reaction-enzymatic reaction with the skin, which increases the content of glycogen and protein, thereby promoting the metabolism and synthesis of cells. The special photons emitted by the LED therapy device can beautify the part of skin of the human body, promote the metabolism and circulation of subcutaneous tissue, increase collagen, accelerate the repair of damaged tissues, relieve pain, accelerate cell renewal and repair, functioning in beauty and therapy.

However, a conventional LED therapy device is made of a single LED light cup, which may may cause poor therapy effect due to a single light emission, and cannot meet consumer's requirements of beauty therapy effects and vision.

### SUMMARY

Accordingly, to overcome the shortcomings of the prior art, the purpose of the present disclosure is to provide a handheld LED therapy device, which solves the problem that the existing portable therapy lamp emit only single light due to its single light cup.

The purpose of the present disclosure can be achieved by the following solutions:

A handheld LED therapy device according to the present disclosure includes a main controlling module and an LED therapy module. The LED therapy module includes a multilight cup comprising a LED therapy light of at least one light color, the main controlling module is connected to the LED therapy light, and the main controlling module can operate a controlling operation to the LED therapy light.

Additionally, the multilight cup includes LED therapy lights of at least two light colors, and the controlling operation comprises simultaneously turn on or turn off the LED therapy lights of at least two light colors, or setting time to the LED therapy light.

Additionally, the LED therapy light of at least one light color has a wavelength ranging from 460 nm to 940 nm.

Additionally, the main controlling module includes a button switch circuit, a battery power supply circuit, a single-chip microcomputer controlling IC circuit, and a switch circuit. The battery power supply circuit can supply electric power, the button switch circuit is electrically connected to the single-chip microcomputer controlling IC circuit, and the single-chip microcomputer controlling IC circuit is connected to the LED therapy light via the switch circuit.

Additionally, the single-chip microcomputer controlling IC includes a single-chip microcomputer and a peripheral circuit thereof, the switch circuit includes a plurality of MOS transistors, and gates of the MOS transistors are connected to a PWM output end of the single-chip microcomputer, drains of the MOS transistors are connected to a power source, and sources of the MOS transistors are connected to the LED therapy light, a resistor is provided between the drains and the sources of the MOS transistors, and an output end of the button switch circuit is connected to a controlling input end of the single-chip microcomputer.

Additionally, the multilight cup includes the LED therapy lights of three light colors, and the controlling operation includes:

the single-chip microcomputer responding to a pressing button operation of a button switch while the handheld LED therapy device is electrified;

step 1: the single-chip microcomputer controlling all of the LED therapy lights to turn on after the button switch being pressed;

step 2: he single-chip microcomputer controlling all of the LED therapy lights to turn off after the button switch being pressed again.

Additionally, the multilight cup includes the LED therapy lights of four light colors, and the controlling operation includes:

the single-chip microcomputer responding to a pressing button operation of a button switch while the handheld LED therapy device is electrified;

step 10: the single-chip microcomputer controlling all of the LED therapy lights to turn on after the button switch being pressed;

step 20: the single-chip microcomputer controlling the LED therapy lights of a first, a second, and a third light colors to turn on, and the LED therapy light of a fourth light color to turn off after the button switch being pressed again; and

step 30: the single-chip microcomputer controlling the LED therapy lights of the first and the second light colors to turn on, and the LED therapy lights of the third and the fourth light color to turn off after the button switch being pressed again; then returning to the step 10.

Additionally, the multilight cup includes the LED therapy lights of five light colors, and the controlling operation includes:

the single-chip microcomputer responding to a pressing button operation of a button switch while the handheld LED therapy device is electrified;

step 100: the single-chip microcomputer controlling all of the LED therapy lights to turn on after the button switch being pressed;

step 200: the single-chip microcomputer controlling the LED therapy lights of a fifth, a sixth, a seventh, and a eighth light colors to turn on, and the LED therapy light of a ninth light color to turn off after the button switch being pressed again;

step 300: the single-chip microcomputer controlling the LED therapy lights of the fifth, the sixth, and the seventh light colors to turn on, and the LED therapy lights of the eighth and the ninth light color to turn off after the button switch being pressed again;

step 400: the single-chip microcomputer controlling the LED therapy lights of the fifth and the sixth light colors to turn on, and the LED therapy lights of the seventh, the eighth, and the ninth light color to turn off after the button switch being pressed again; and

step 500: the single-chip microcomputer controlling the LED therapy lights of the fifth and the seventh light colors to turn on, and the LED therapy lights of the sixth, the eighth, and the ninth light color to turn off after the button switch being pressed again; then returning to the step 100.

Additionally, the controlling operation further includes: while at least one of the LED therapy lights is turned on, the single-chip microcomputer controlling controlling at least one of the LED therapy lights to flash in response of a long-press operation of the button switch.

Additionally, the battery power supply circuit includes a hollow battery barrel, and a battery sleeve for mounting a battery is provided in the battery barrel, an anode electrode sheet and a cathode spring are provided on both ends of the battery sleeve, respectively. The side of the anode electrode sheet adjacent to to battery is provided with an anode insulation sheet, a side of the anode insulation sheet adjacent to the battery is provided with a pressing ring, and the switch circuit is provided on a side of the anode insulation sheet away from the battery, a lamp cap is located on an end of the battery sleeve, and the multilight cup and the switch circuit are both located in the lamp cap, the button switch circuit comprises a button mounted on a switch panel via a switch positioning sheet, the switch panel is fixed on a positioning block, and both sides of the positioning block abut to the battery sleeve and a controlling panel, respectively. The controlling panel is provided with a controlling block, the single-chip microcomputer controlling IC circuit is mounted in the controlling block, a light panel is provided on a side of the controlling block away from the battery, the multilight cup is mounted on the light panel via an insulation pad, a lens is arranged on a light emitting direction of the multilight cup, and a light emitted from the multilight cup passes through the lens, and is emitted from a side of the lamp cup away from the battery.

Compared to the prior art, the present disclosure has the following beneficial effects:

The present disclosure includes a main controlling module and an LED therapy module. The LED therapy module includes a multilight cup comprising a LED therapy light of at least one light color, the main controlling module is connected to the LED therapy light, and the main controlling module can operate a controlling operation to the LED therapy light, such that the light of at least one color can beautify the part of skin of the human body, promote the metabolism and circulation of subcutaneous tissue, increase collagen, accelerate the repair of damaged tissues, relieve pain, accelerate cell renewal and repair, functioning in beauty and therapy. The device of the present invention is very portable, simple in structure, compact in size, and the light output from the multilight cup is larger, and the therapy effect is better, which can meet consumers' requirements of beauty therapy effects and vision.

### BRIEF DESCRIPTION OF THE DRAWINGS

FIG. 1 is a schematic view of a handheld LED therapy device according to an embodiment of the present disclosure;

FIG. 2a is a schematic view of a button switch circuit according to an embodiment of the present disclosure;

FIG. 2b is a schematic view of a battery power supply circuit according to an embodiment of the present disclosure;

FIG. 2c is a schematic view of a single-chip microcomputer controlling IC according to an embodiment of the present disclosure;

FIG. 2d is a schematic view of a switch circuit according to an embodiment of the present disclosure;

FIG. 3a is a cross-sectional view of a handheld LED therapy device according to an embodiment of the present disclosure;

FIG. 3b is an exploded view of a handheld LED therapy device according to an embodiment of the present disclosure;

FIG. 3c is a perspective view of a handheld LED therapy device according to an embodiment of the present disclosure;

FIG. 4a is a side view of a multilight cup including a LED therapy lights of three light colors according to an embodiment of the present disclosure in a first angle of view;

FIG. 4b is a cross-sectional view of the multilight cup including the LED therapy lights of three light colors according to an embodiment of the present disclosure in a second angle of view;

FIG. 5a is a cross-sectional view of a multilight cup including a LED therapy lights of four light colors according to an embodiment of the present disclosure in the first angle of view;

FIG. 5b is a cross-sectional view of the multilight cup including the LED therapy lights of four light colors according to an embodiment of the present disclosure in the second angle of view;

FIG. 6a is a cross-sectional view of a multilight cup including a LED therapy lights of five light colors according to an embodiment of the present disclosure in the first angle of view; and

FIG. 6b is a cross-sectional view of the multilight cup including the LED therapy lights of five light colors according to an embodiment of the present disclosure in the second angle of view.

### DETAILED DESCRIPTION OF THE EMBODIMENTS

The present disclosure will be further described in detail below with reference to the drawings and detailed embodiments.

### Embodiment 1

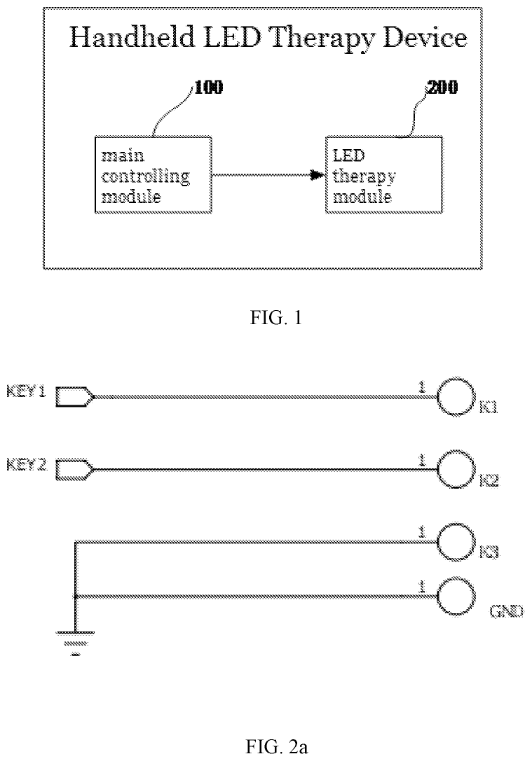

Referring to FIG. 1, a handheld LED therapy device according to the present disclosure includes a main controlling module 100 and an LED therapy module 200. The LED therapy module 100 includes a multilight cup 3, the multilight cup 3 includes a LED therapy light of at least one light color, the main controlling module 100 is connected to the LED therapy light, and the main controlling module 100 can operate a controlling operation to the LED therapy light.

Specifically, the controlling operation can be at least one of the following commands: Controlling the LED therapy module 200 to turn on or turn off, adjusting the pulse of the LED therapy module 200, adjusting the frequency of flash of the LED therapy module 200, setting time to the LED therapy module 200, switching the color of light of the LED therapy module 200, and adjusting the LED therapy module 200 to be indirectly flashed.

The multilight cup 3 may include the LED therapy light of single light color, or the LED therapy lights of at least two light colors. For example, it can include the LED therapy lights of three, four, five, or more light colors.

Specifically, if the multilight cup 3 includes LED therapy lights of at least two light colors, the controlling operation further includes: simultaneously turn on or turn off the LED therapy lights of at least two light colors, or setting time to the LED therapy light. Namely, the device has a timing function, for example, the device will automatically shut down after a few minutes of continuous operation.

If the multilight cup 3 includes LED therapy lights of three, four, five, or more light colors, all or part of the the LED therapy lights of three, four, five, or more light colors can be turned on or off at the same time, or the LED therapy lights can be timed.

Optionally, the LED therapy light of at least one light color has a wavelength ranging from 460 nm to 940 nm. When the wavelength of the LED therapy light ranges from 460 nm to 940 nm, the photons of the specified color of light can beautify the part of skin of the human body, promote the metabolism and circulation of subcutaneous tissue, increase collagen, accelerate the repair of damaged tissues, relieve pain, accelerate cell renewal and repair, bringing a better beauty and therapy effect, which can be functioned in the therapy of human and animal bodies. Specifically, the wavelength of the light can be 630 nm, 660 nm, 760 nm, 830 nm, or 850 nm.

Optionally, the main controlling module 100 includes a button switch circuit, a battery power supply circuit, a single-chip microcomputer controlling IC circuit, and a switch circuit. The battery power supply circuit can supply electric power, the button switch circuit is electrically connected to the single-chip microcomputer controlling IC circuit, and the single-chip microcomputer controlling IC circuit is connected to the LED therapy light via the switch circuit.

Optionally, the single-chip microcomputer controlling IC includes a single-chip microcomputer and a peripheral circuit thereof, the switch circuit includes a plurality of MOS transistors, and gates of the MOS transistors is connected to a PWM output end of the single-chip microcomputer, drains of the MOS transistors are connected to a power source, and sources of the MOS transistors are connected to the LED therapy light, a resistor is provided between the drains and the sources of the MOS transistors, and an output end of the button switch circuit is connected to a controlling input end of the single-chip microcomputer.

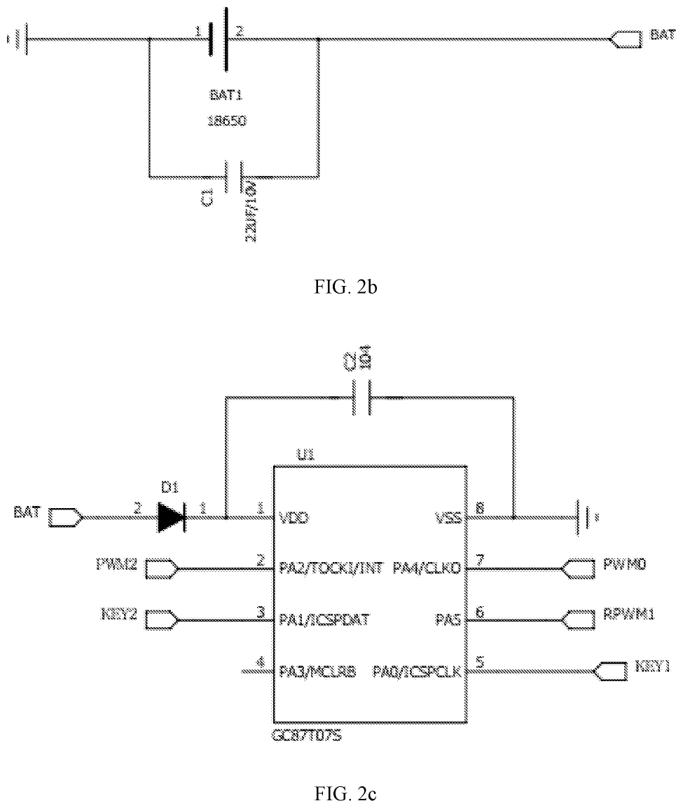

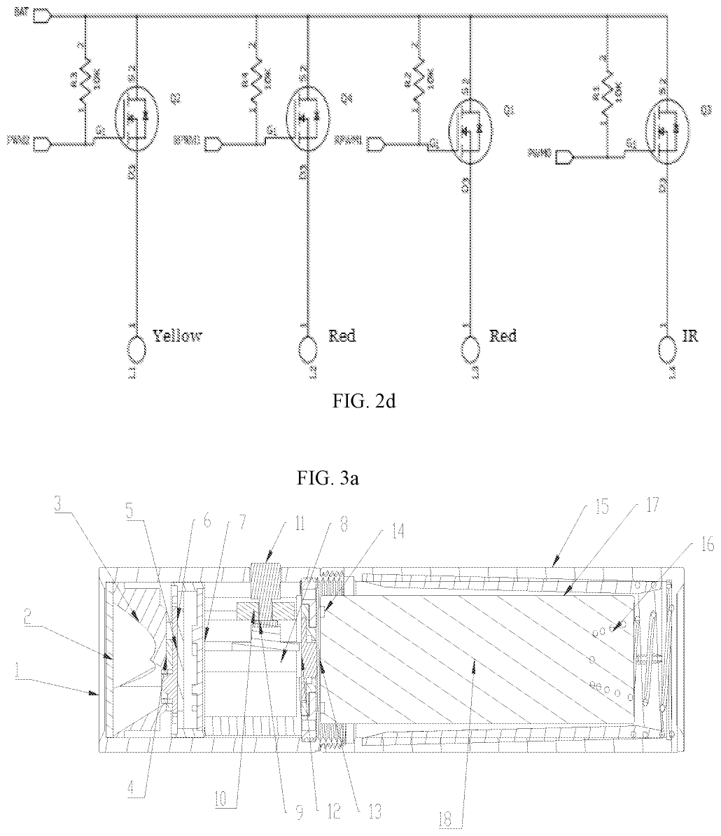

Referring to FIGS. 2a, 2b, 2c, and 2d, a button switch circuit, a battery power supply circuit, a single-chip microcomputer controlling IC circuit, and a switch circuit are shown. The single-chip microcomputer controlling IC includes a GC87T07S chip and a peripheral circuit thereof. The switch circuit includes a plurality of MOS transistors, and gates of the MOS transistors Q1, Q2, Q3, and Q4 are connected to a PWM output end of the GC87T07S chip, drains of the MOS transistors Q1, Q2, Q3, and Q4 are connected to a power source, and sources of the MOS transistors Q1, Q2, Q3, and Q4 are connected to the LED therapy light of red, LED therapy light of yellow, the LED therapy light of red, and the LED therapy light of infrared light, respectively. Resistors R1, R2, R3, and R4 are located between the drains and the sources of the MOS transistors Q1, Q2, Q3, and Q4, and the first and the second ends of the button switch circuit are connected to the third and the fifth pins of the GC87T07S chip.

Optionally, if the multilight cup 3 includes the LED therapy lights of three light colors, and the controlling operation includes:

the single-chip microcomputer responding to a pressing button operation of a button switch while the handheld LED therapy device is electrified;

step 1: the single-chip microcomputer controlling all of the LED therapy lights to turn on after the button switch being pressed;

step 2: he single-chip microcomputer controlling all of the LED therapy lights to turn off after the button switch being pressed again.

Optionally, if the multilight cup 3 includes the LED therapy lights of four light colors, and the controlling operation includes:

the single-chip microcomputer responding to a pressing button operation of a button switch while the handheld LED therapy device is electrified;

step 10: the single-chip microcomputer controlling all of the LED therapy lights to turn on after the button switch being pressed;

step 20: the single-chip microcomputer controlling the LED therapy lights of a first, a second, and a third light colors to turn on, and the LED therapy light of a fourth light color to turn off after the button switch being pressed again; and

step 30: the single-chip microcomputer controlling the LED therapy lights of the first and the second light colors to turn on, and the LED therapy lights of the third and the fourth light color to turn off after the button switch being pressed again; then returning to the step 10.

Optionally, if the multilight cup 3 includes the LED therapy lights of five light colors, and the controlling operation includes:

the single-chip microcomputer responding to a pressing button operation of a button switch while the handheld LED therapy device is electrified;

step 100: the single-chip microcomputer controlling all of the LED therapy lights to turn on after the button switch being pressed;

step 200: the single-chip microcomputer controlling the LED therapy lights of a fifth, a sixth, a seventh, and a eighth light colors to turn on, and the LED therapy light of a ninth light color to turn off after the button switch being pressed again;

step 300: the single-chip microcomputer controlling the LED therapy lights of the fifth, the sixth, and the seventh light colors to turn on, and the LED therapy lights of the eighth and the ninth light color to turn off after the button switch being pressed again;

step 400: the single-chip microcomputer controlling the LED therapy lights of the fifth and the sixth light colors to turn on, and the LED therapy lights of the seventh, the eighth, and the ninth light color to turn off after the button switch being pressed again; and

step 500: the single-chip microcomputer controlling the LED therapy lights of the fifth and the seventh light colors to turn on, and the LED therapy lights of the sixth, the eighth, and the ninth light color to turn off after the button switch being pressed again; then returning to the step 100.

Optionally, the controlling operation further includes: while at least one of the LED therapy lights is turned on, the single-chip microcomputer controlling controlling at least one of the LED therapy lights to flash in response of a long-press operation of the button switch.

The special photons emitted by the LED therapy device can beautify the part of skin of the human body, promote the metabolism and circulation of subcutaneous tissue, increase collagen, accelerate the repair of damaged tissues, relieve pain, accelerate cell renewal and repair, functioning in beauty and therapy.

### Embodiment 2

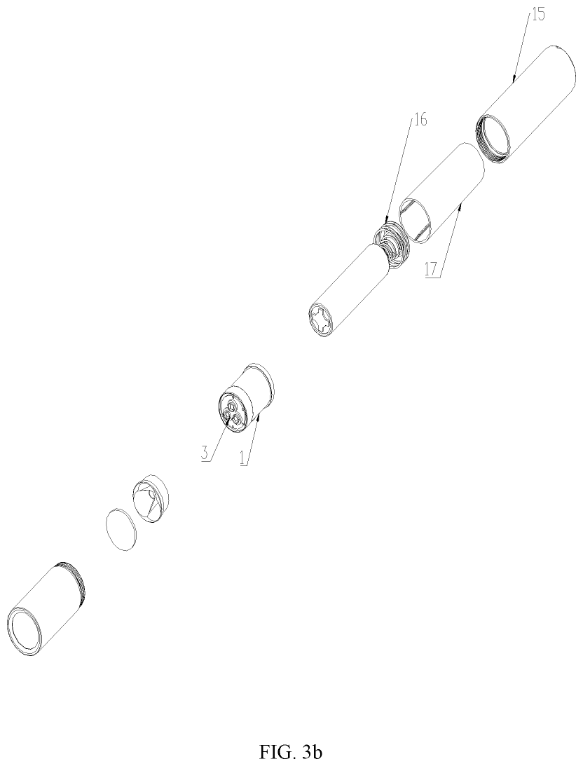

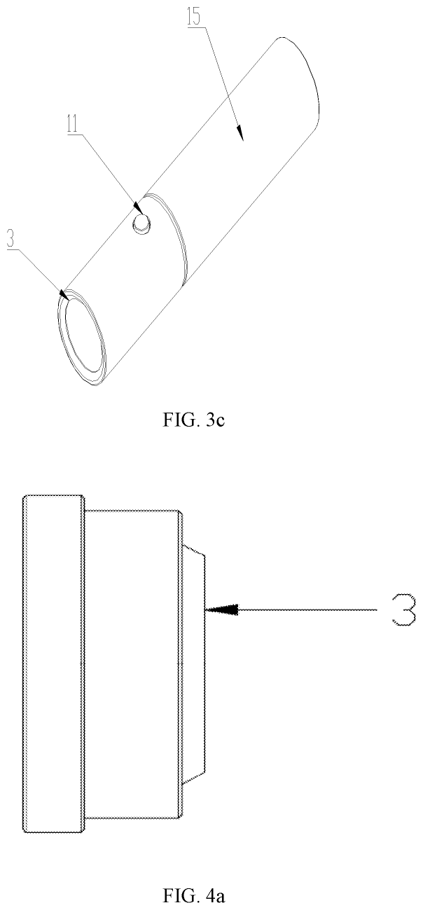

As a detailed embodiment, the cross-sectional view, the exploded view, and the perspective view can be shown in FIGS. 31, 3b, and 3c. The battery power supply circuit includes a hollow battery barrel 15, and a battery sleeve 17 for mounting a battery 18 is provided in the battery barrel. An anode electrode sheet 12 and a cathode spring 16 are located on both ends of the battery sleeve 17, respectively. The side of the anode electrode sheet 12 adjacent to to battery 18 is provided with an anode insulation sheet 13, a side of the anode insulation sheet 13 adjacent to the battery 18 is provided with a pressing ring 14, and the switch circuit is located on a side of the anode insulation sheet 13 away from the battery 18. A lamp cap 1 is located on an end of the battery sleeve 15, and the multilight cup 3 and the switch circuit are both located in the lamp cap 1. The button switch circuit includes a button 11 mounted on a switch panel 9 via a switch positioning sheet 10, the switch panel 9 is fixed on a positioning block 8, and both sides of the positioning block 8 abut to the battery sleeve 15 and a controlling panel 7, respectively. The controlling panel 7 is mounted with a controlling block 6, the single-chip microcomputer controlling IC circuit is mounted in the controlling block 6, a light panel 5 is arranged on a side of the controlling block 6 away from the battery 18, the multilight cup 3 is mounted on the light panel 5 through an insulation pad 4, a lens 2 is arranged on a light emitting direction of the multilight cup 3, and the light emitted from the multilight cup 3 passes through the lens 2, and is emitted from a side of the lamp cup 1 away from the battery 18.

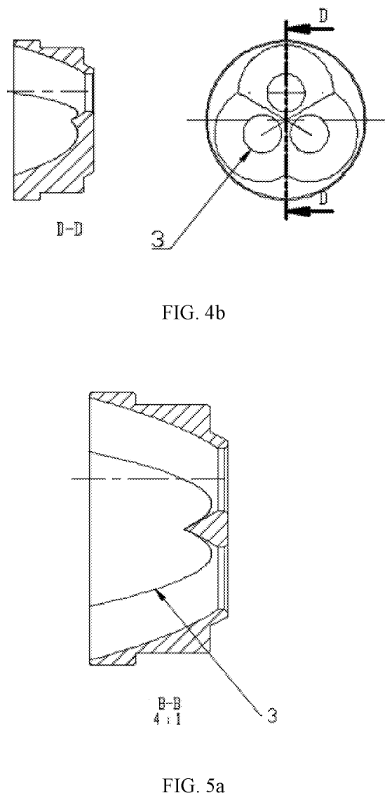

FIGS. 4a and 4b are cross-sectional views of a multilight cup including a LED therapy lights of three light colors according to an embodiment of the present disclosure in a first and a second angle of view. The multilight cup 3 includes three light cups, and each of the light cups has a LED therapy light inside, and the three light cups are arranged as a ring in the lamp cup 1.

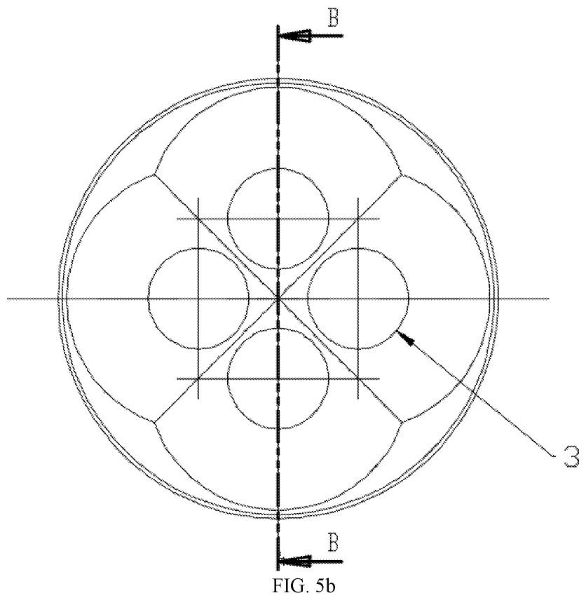

FIGS. 5a and 5b are cross-sectional views of a multilight cup including a LED therapy lights of four light colors according to an embodiment of the present disclosure in the first and second angles of view. The multilight cup 3 includes four light cups, and each of the light cups has a LED therapy light inside, and the four light cups are arranged as a ring in the lamp cup 1.

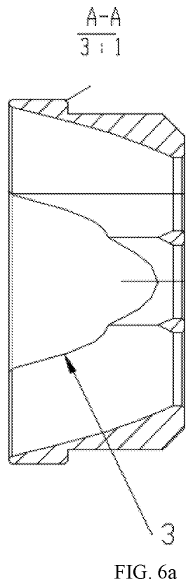

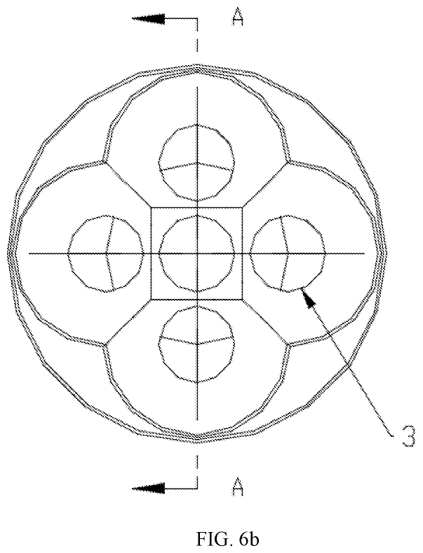

FIGS. 6a and 6b are cross-sectional views of a multilight cup including a LED therapy lights of four light colors according to an embodiment of the present disclosure in the first and second angles of view. The multilight cup 3 includes five light cups, and each of the light cups has a LED therapy light inside, and the five light cups are arranged as a ring in the lamp cup 1.

According to the above solutions, the lights of three, four, or five colors can be combined, such that the lights of three, four, or five colors can be unified. Compared to a single-color light cup, the light output is larger, and the therapy effect is better, which can meet consumers' requirements of beauty therapy effects and vision.

The method of controlling operation in the embodiments of the present disclosure can also be implemented through a computer storage medium. When implemented through a computer storage medium, a computer program is stored thereon. If the method of controlling operation of the present disclosure is implemented in the form of a software functional unit and is sold or used as a independent product, it can be stored in the computer storage medium. When the computer program is executed by the processor, it can implement the steps of the above embodiments. Wherein, the computer program includes computer program code, and the computer program code may be in the form of source code, object code, executable file, or some intermediate forms. The computer storage medium may include: any entity or device capable of carrying the computer program code, recording medium, U disk, mobile hard disk, magnetic disk, optical disk, computer memory, read-only memory (ROM, Read-Only Memory), Random Access Memory (RAM, Random Access Memory), electric carrier signal, telecommunications signal, software distribution medium, etc. It should be noted that, the content included in the computer storage medium can be appropriately added or deleted according to the requirements of the legislation and patent practice in the jurisdictions. For example, in some jurisdictions, according to the legislation and patent practice, the computer storage medium does not include electric carrier signal and telecommunication signal.

The specific embodiments are only explanations of the present disclosure, and the embodiments are not intended to limit the present disclosure. Rather, various modifications may be made in the details within the scope and range of equivalents of the claims and without departing from the present disclosure. The scope of this present disclosure is not limited to the content of the description, and its technical scope shall be determined according to the scope of the claims.

What is claimed is:

1. A handheld LED therapy device, comprising a main controlling module and an LED therapy module, wherein the LED therapy module comprises a multilight cup comprising a LED therapy light of at least one light color, the main controlling module is connected to the LED therapy light, and the main controlling module is capable of operating a controlling operation to the LED therapy light.

2. The handheld LED therapy device according to claim 1, wherein the multilight cup comprises LED therapy lights of at least two light colors, and the controlling operation comprises simultaneously turn on or turn off the LED therapy lights of at least two light colors, or setting time to the LED therapy light.

3. The handheld LED therapy device according to claim 1, wherein the LED therapy light of at least one light color has a wavelength ranging from 460 nm to 940 nm.

4. The handheld LED therapy device according to claim 1, wherein the main controlling module comprises a button switch circuit, a battery power supply circuit, a single-chip microcomputer controlling IC circuit, and a switch circuit; the battery power supply circuit is capable of supplying electric power, the button switch circuit is electrically connected to the single-chip microcomputer controlling IC circuit, and the single-chip microcomputer controlling IC circuit is connected to the LED therapy light via the switch circuit.

5. The handheld LED therapy device according to claim 4, wherein the single-chip microcomputer controlling IC comprises a single-chip microcomputer and a peripheral circuit thereof, the switch circuit comprises a plurality of MOS transistors, and gates of the MOS transistors are connected to a PWM output end of the single-chip microcomputer, drains of the MOS transistors are connected to a power source, and sources of the MOS transistors are connected to the LED therapy light, a resistor is provided between the drains and the sources of the MOS transistors, and an output end of the button switch circuit is connected to a controlling input end of the single-chip microcomputer.

6. The handheld LED therapy device according to claim 5, wherein the multilight cup comprises the LED therapy lights of three light colors, and the controlling operation comprises:
the single-chip microcomputer responding to a pressing button operation of a button switch while the handheld LED therapy device is electrified;
step 1: the single-chip microcomputer controlling all of the LED therapy lights to turn on after the button switch being pressed;
step 2: he single-chip microcomputer controlling all of the LED therapy lights to turn off after the button switch being pressed again.

7. The handheld LED therapy device according to claim 5, wherein the multilight cup comprises the LED therapy lights of four light colors, and the controlling operation comprises:
the single-chip microcomputer responding to a pressing button operation of a button switch while the handheld LED therapy device is electrified;
step 10: the single-chip microcomputer controlling all of the LED therapy lights to turn on after the button switch being pressed;
step 20: the single-chip microcomputer controlling the LED therapy lights of a first, a second, and a third light colors to turn on, and the LED therapy light of a fourth light color to turn off after the button switch being pressed again; and
step 30: the single-chip microcomputer controlling the LED therapy lights of the first and the second light colors to turn on, and the LED therapy lights of the third and the fourth light color to turn off after the button switch being pressed again; then returning to the step 10.

8. The handheld LED therapy device according to claim 5, wherein the multilight cup comprises the LED therapy lights of five light colors, and the controlling operation comprises:
the single-chip microcomputer responding to a pressing button operation of a button switch while the handheld LED therapy device is electrified;
step 100: the single-chip microcomputer controlling all of the LED therapy lights to turn on after the button switch being pressed;
step 200: the single-chip microcomputer controlling the LED therapy lights of a fifth, a sixth, a seventh, and a eighth light colors to turn on, and the LED therapy light of a ninth light color to turn off after the button switch being pressed again;
step 300: the single-chip microcomputer controlling the LED therapy lights of the fifth, the sixth, and the seventh light colors to turn on, and the LED therapy lights of the eighth and the ninth light color to turn off after the button switch being pressed again;
step 400: the single-chip microcomputer controlling the LED therapy lights of the fifth and the sixth light colors to turn on, and the LED therapy lights of the seventh, the eighth, and the ninth light color to turn off after the button switch being pressed again; and
step 500: the single-chip microcomputer controlling the LED therapy lights of the fifth and the seventh light colors to turn on, and the LED therapy lights of the sixth, the eighth, and the ninth light color to turn off after the button switch being pressed again; then returning to the step 100.

9. The handheld LED therapy device according to claim 7, wherein the controlling operation further comprises: while at least one of the LED therapy lights is turned on, the single-chip microcomputer controlling controlling at least one of the LED therapy lights to flash in response of a long-press operation of the button switch.

10. The handheld LED therapy device according to claim 4, wherein the battery power supply circuit comprises a hollow battery barrel, and a battery sleeve for mounting a battery is provided in the battery barrel, an anode electrode sheet and a cathode spring are provided on both ends of the battery sleeve, respectively; a side of the anode electrode sheet adjacent to to battery is provided with an anode insulation sheet, a side of the anode insulation sheet adjacent to the battery is provided with a pressing ring, and the switch circuit is provided on a side of the anode insulation sheet away from the battery, a lamp cap is located on an end of the battery sleeve, and the multilight cup and the switch circuit are both located in the lamp cap, the button switch circuit comprises a button mounted on a switch panel via a switch positioning sheet, the switch panel is fixed on a positioning block, and both sides of the positioning block abut to the battery sleeve and a controlling panel, respectively; the controlling panel is provided with a controlling block, the single-chip microcomputer controlling IC circuit is mounted in the controlling block, a light panel is provided on a side of the controlling block away from the battery, the multilight cup is mounted on the light panel via an insulation pad, a lens is arranged on a light emitting direction of the multilight cup, and a light emitted from the multilight cup passes through the lens, and is emitted from a side of the lamp cup away from the battery.
